(19) United States
(12) Patent Application Publication
(10) Pub. No.: US 2018/0251769 A1
(43) Pub. Date: Sep. 6, 2018

(54) Recombinant Influenza Virus

(71) Applicant: The Regents of the University of California, Oakland, CA (US)

(72) Inventor: Genhong Cheng, Los Angeles, CA (US)

(21) Appl. No.: 15/908,723

(22) Filed: Feb. 28, 2018

Related U.S. Application Data
(60) Provisional application No. 62/635,692, filed on Feb. 27, 2018.

(30) Foreign Application Priority Data
Mar. 1, 2017 (CN) 201710115724
Mar. 1, 2017 (CN) 201710118851

(51) Int. Cl. C12N 15/113; C12N 7/06; C07K 14/11

(52) U.S. Cl. CPC C12N 15/1131; C12N 7/06; C12N 2760/16162; C12N 2760/16262; C07K 14/11

(57) ABSTRACT

Disclosed herein are recombinantly engineered influenza viruses and compositions thereof.

### CROSS REFERENCE TO RELATED APPLICATION DATA

This application claims the benefit of U.S. Application No. 62/635,692, filed Feb. 27, 2018, and priority to CN 201710115724, filed Mar. 1, 2017, and CN 201710118851, filed Mar. 1, 2017, which are all herein incorporated by reference in their entirety. This application is also related to PCT/CN2017/080560 and PCT/CN2017/080561, both of which are herein incorporated by reference in their entirety.

### REFERENCE TO A SEQUENCE LISTING SUBMITTED VIA EFS-WEB

The content of the ASCII text file of the sequence listing named “20180228_034044_180_seq_ST25” which is 8.94 kb in size was created on Feb. 28, 2018 and electronically submitted via EFS-Web herewith the application is incorporated herein by reference in its entirety.

### BACKGROUND OF THE INVENTION

### 1. Field of the Invention

The present invention relates to the field of viruses, particularly recombinantly engineered influenza viruses and its applications.

### 2. Description of the Related Art

Generally, the genomes of Influenzavirus comprise seven to eight segments of negative-sense single-strand RNA (ssRNA(−)), which encode 7 to 14 proteins. Influenzavirus belongs to the family of Orthomyxoviridae family. To date, there are 4 recognized genera of Influenzavirus: Influenzavirus A, Influenzavirus B, Influenzavirus C, and Influenzavirus D. Influenza A virus is the only known species of the Influenzavirus A genus, Influenza B virus is the only known species of the Influenzavirus B genus, Influenza C virus is the only known species of the Influenzavirus C genus, and Influenza D virus is the only known species of the Influenzavirus D genus. The Influenzavirus A genome consists of 8 segments and encodes 12-14 proteins depending on the given strain. The Influenzavirus B genome consists of 8 segments and encodes 11 proteins. The Influenzavirus C genome consists of 7 segments and encodes 9 proteins. Influenzavirus D is closely related to Influenzavirus C, and its genome similarly consists of 7 segments.

The genome segments are numbered according to molecular weight in descending order. For Influenza A virus, the eight segments encode PB2, PB1, PA, HA, NP, NA, M, and NS proteins and their alternative splice variants. Hemagglutinin (HA) and neuraminidase (NA) are major surface antigens of Influenza A virus and the differences between these surface antigens are used to divide Influenza A virus into subtypes. For example, the H5N1 virus designates an influenza A subtype that has a type 5 hemagglutinin (H) protein and a type 1 neuraminidase (N) protein. There are 18 known types of hemagglutinin and 11 known types of neuraminidase. Thus, 198 different combinations of these proteins are possible.

Influenza A virus and Influenza B virus are common among human populations and can cause a worldwide pandemic, but the extent of Influenza A virus infection is greater and more dangerous. Influenza viruses can cause severe respiratory diseases. They are highly contagious and easily cause other serious complications. Multiple global pandemics have occurred, resulting in great harm to human lives and health. Since the viral surface proteins HA and NA are prone to mutate, they can produce two forms of mutations, including antigenic drift and antigenic shift. In recent years, outbreaks and epidemics of influenza virus mutants such as H1N1 and H7N9 in the world and China have caused more difficulties in terms of disease control against a background of the global integration. With increasingly frequent interactions, the frequency of recombination or re-assortment of viruses has also increased. This has not only made it even more difficult to predict new mutant strains but has also resulted in huge economic losses for various countries and regions, brought huge stress to human health and lives, and made efforts to control the disease more difficult.

Currently, vaccination is the most effective means to prevent pandemics of influenza virus. The flu vaccine currently in wide use is designed to target HA and NA proteins in the virus and uses HA and NA proteins to induce the body to generate protective immunity by using HA and NA as the target antigens. The inactivated virus vaccines that have been currently approved for use in the human body are two Influenza A viruses (H1N1 and H3N2), which pose greater harm to humans, and a trivalent inactivated vaccine consisting of Influenza B virus. Although the inactivated whole virus vaccine designed for HA and NA it is quite safe, has complete antigenic components and strong immunogenic characteristics, is resistant to identical subtypes of Influenza A viruses and provides good immune protection, the current vaccine does not provide a very ideal prevention and control result during the epidemic and outbreak of an influenza virus. On the one hand, because of the important role played by the characteristics of the virus itself, while at the same time, the design methods, research and development strategy and protective effect, etc., of the vaccine also have a direct impact. First, HA and NA proteins in influenza virus are prone to an antigenic drift or shift. The production of new vaccines for pandemic strains should be updated in a timely manner together with the mutation of epidemic virus strains. The breeding of virus vaccine strains is time-consuming and laborious, with a long production cycle and a high cost, which makes it difficult to adapt to the need to prevent and control influenza pandemics. Second, it is difficult for an inactivated vaccine to provide adequate immune protection against a virus infection as it cannot effectively stimulate a cellular immune response. Therefore, designing an appropriate target antigen by choosing a vaccine to accelerate efficient screening of candidate vaccine strains and provide adequate improvement of the immune protection effect of vaccines is a critical issue in current influenza vaccine research that needs to be addressed urgently.

Among the choices of target antigens for Influenza A vaccine development, apart from virus surface proteins HA and NA, matrix protein M has also been widely reported by many scholars. M protein is encoded by virus RNA segment 7. It contains 1027 nucleotides, including non-sugar glycosylated structural proteins M1 and M2. M1 and M2 coding regions partially overlap but have a different open reading frame. The M1 protein comprises 252 amino acids. The M2 protein coding region comprises nucleotides 26˜51 and 740˜1007 and encodes 97 amino acids. The M1 forms a dimer, binding with viral RNA and capsule and plays a role in viral nucleocapsid assembly. The M1 has a low mutation rate, is type-specific and its antigenic differences are part of the basis for virus typing. The M2 protein is one of the membrane proteins of the influenza virus and is expressed in low density on the Influenza A virus membrane. It is widely distributed on the infected cell membrane. M2 protein is present in the form of a homotetramer on the lipid membrane and plays the role of a proton pump. By controlling the activity of the proton channel, it adjusts the pH within the virus, thus affecting the replication of the influenza virus. Since the M2 protein is the third transmembrane in addition to HA and NA, it is highly conservative in human Influenza A virus. The M2 protein has become a hot topic for the study of universal influenza virus vaccines.

Live attenuated influenza vaccine (LAIV) can stimulate both humoral and cellular immunity and is therefore a primary focus in flu vaccine research and development. Live attenuated influenza vaccine has more advantages compared with the inactivated vaccine. The immunization route of live attenuated vaccine is similar to natural infection by virus. Respiratory tract replication can induce an effective mucosal immune response, generate a large amount of secretory IgA, induce a strong cellular and humoral immune response to effectively control the propagation of respiratory virus; the drug can be intranasally administered by spray or nasal route, which is very convenient, thus avoiding the problems associated with the route of injection; cellular immunity and slgA antibodies induced by transnasal attenuated vaccine have some cross-protection functions against different subtypes of influenza virus.

Traditional design methods for a live attenuated vaccine repeatedly choose a forward genetics approach to perform screenings of live candidate vaccines for mutations under non-physiological conditions and can only produce a small amount of candidate vaccine strains. The process of preparing attenuated cold-adapted influenza virus vaccines is complicated time-consuming, laborious, and technically demanding. Currently, reverse genetics technology is used to separately clone 6 genes from cold-adapted virus strains and 2 HA and NA genes from the epidemic virus strains in the current year into 8 plasmids, where they co-transfected mammalian cells. This simplifies the preparation process of cold-adapted attenuated live vaccines and speed up of vaccine development. However, the reverse genetic technology still cannot be used for large-scale screenings of candidate strains of attenuated live vaccines and it is difficult for the process to adapt to the current need to prevent and control the epidemic of influenza. If the new technological method is used in combination with reverse genetic operations to accelerate the screenings of candidate vaccine strains on a large scale, it will provide a new research direction for the designs of the current attenuated live influenza vaccine and also provides reference for the design and development of the virus vaccines that have a manipulation platform of reverse genetics.

### SUMMARY OF THE INVENTION

In some embodiments, the present invention is directed to a recombinantly engineered influenza virus which comprises an insertional mutation in a genomic segment that encodes an M2 protein as disclosed herein, e.g., according to any one of paragraphs [00045] to [0054].

In some embodiments, the present invention is directed to a composition which comprises, consists essentially of, or consists of one or more recombinantly engineered influenza viruses as disclosed herein, e.g., according to any one of paragraphs [0045] to

In some embodiments, the composition further comprises a pharmaceutically acceptable carrier and/or an adjuvant.

In some embodiments, the present invention is directed to a kit which comprises, consists essentially of, or consists of one or more recombinantly engineered influenza viruses as disclosed herein, e.g., according to any one of paragraphs [0045] to [0054], packaged together with a drug delivery device, one or more mutated M2 proteins or compositions thereof, and/or one or more antibodies that specifically bind a mutated M2 protein or a recombinantly engineered influenza virus as disclosed herein. In some embodiments, the present invention is directed to a kit which comprises, consists essentially of, or consists of several doses, each provided in an individual container, of one or more recombinantly engineered influenza viruses packaged together.

In some embodiments, the present invention is directed to a method of inducing an immune response in a subject which comprises administering to the subject an immunogenic amount of one or more recombinantly engineered influenza viruses as disclosed herein, e.g., according to any one of paragraphs [0045] to [0054].

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1, Panel B, is a comparison aligning the M2 nucleic acid coding sequences of the wild-type WSN virus (WT-M2) and the mutated virus (W7-791) containing an insertional mutation. The WT-M2 sequence is SEQ ID NO: 1, the W7-791 sequence is SEQ ID NO: 3, and the insertional mutation is SEQ ID NO: 5.

FIG. 1, Panel B, is a comparison aligning the M2 amino acid sequences of the wild-type WSN virus (WT-M2) and the mutated virus (W7-791) containing an insertional mutation. The WT-M2 sequence is SEQ ID NO: 2, the W7-791 sequence is SEQ ID NO: 4, and the insertional mutation is SEQ ID NO: 6.

FIG. 2 schematically shows the position of the mutation insertion of W7-791 mapped onto the known crystal structure of the M2 protein. A better version of this figure is FIG. 1C of Wang, et al. (2017) Cell Host & Microbe 21: 334-343, which is herein incorporated by reference in its entirety.

FIG. 3 Determination of virus titers. (With 0.25 MOI WSN wild-type virus and virus-infected MDCK cells W7-791 to detect viral titer at different time points).

FIG. 4 influence of W7-791 infection on the survival of MDCK cells.

FIG. 5 Evaluation of the effect of immunization of virus W7-791.

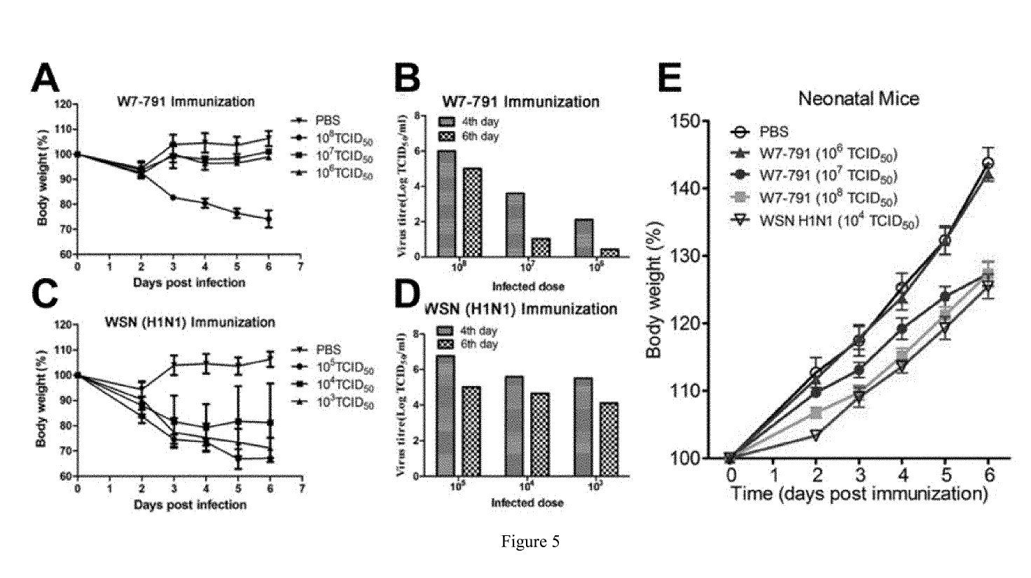

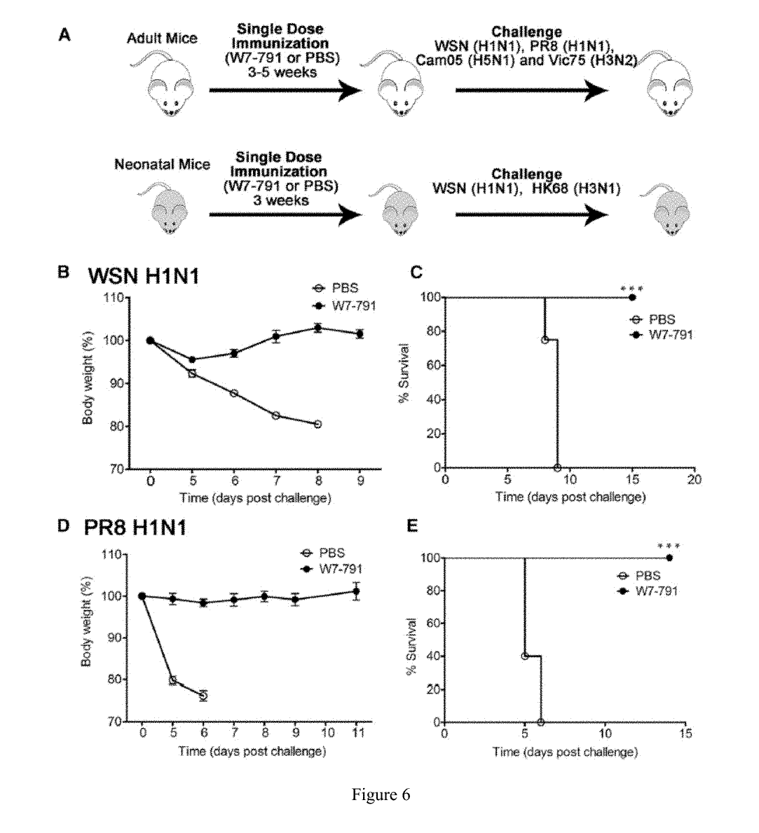

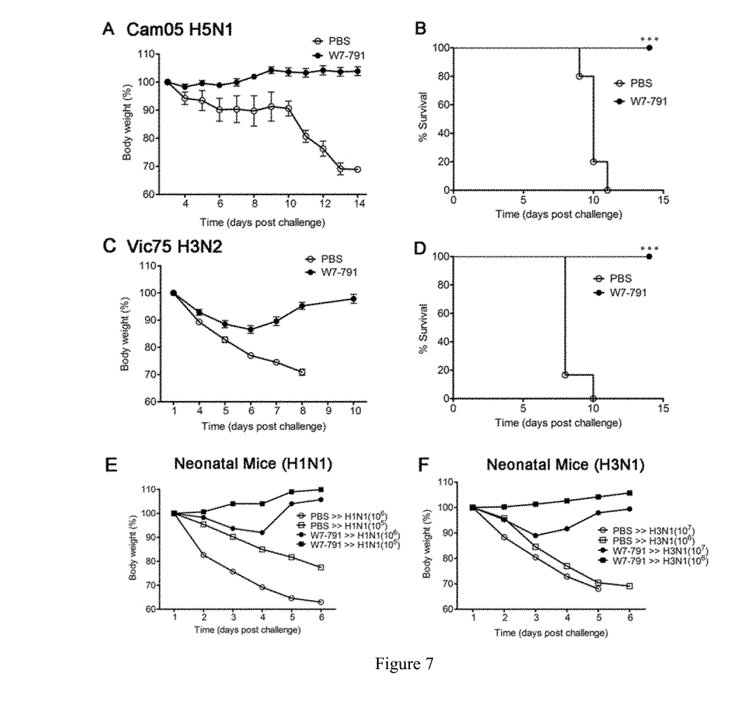

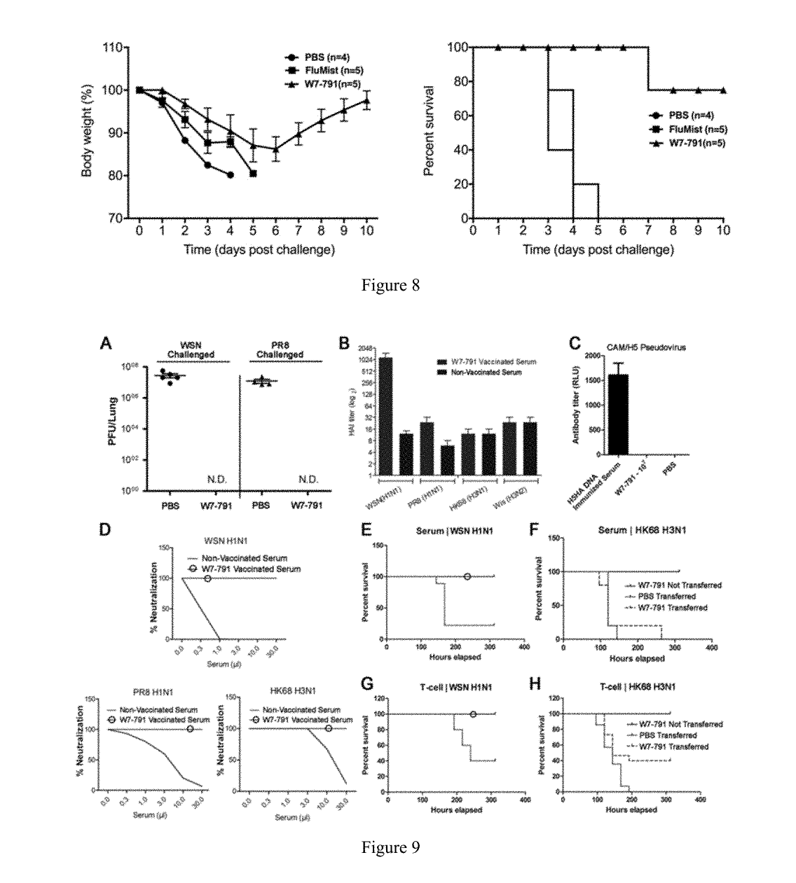

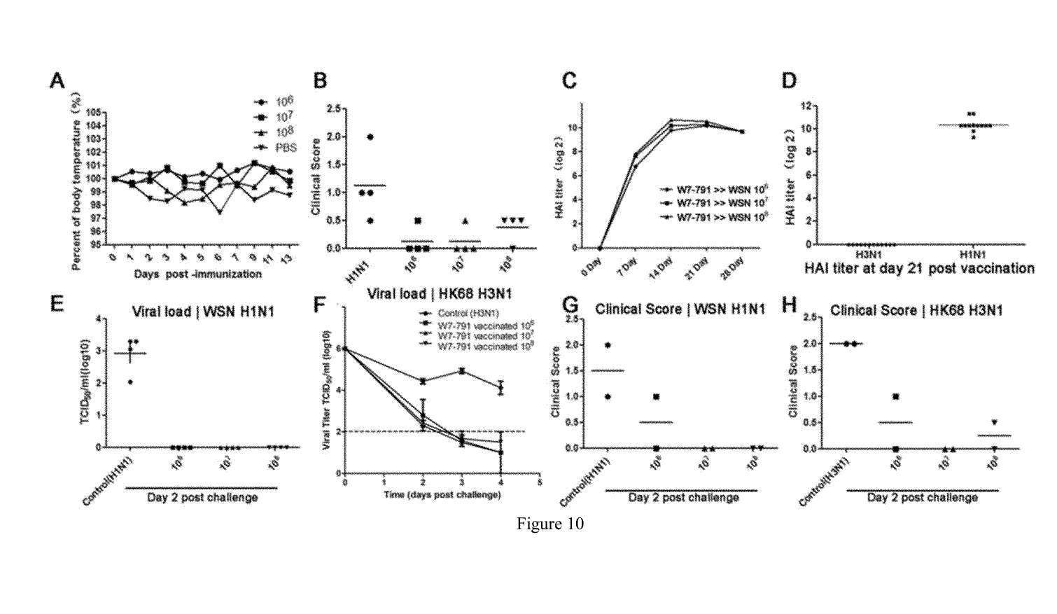

In FIG. 5, weight of mice that have been inoculated with (Panels A, C) W7-791 or WSN wild-type virus of 10⁶, 10⁷ or 10⁸ TCID₅₀ is determined; (Panels B, D) the virus titers on the fourth day and sixth day after inoculation is determined; and (Panel E) the weight of newborn BALB/c mice inoculated with W7-791, WSN or PBS was monitored.

In FIG. 6, a single immunization with W7-791 can activate protection against a lethal dose of influenza virus infection.

In FIG. 6, (Panel A) is a schematic diagram of the immunization and virus infection process of mice; (Panels B-C) each group of 5 mice intranasally immunized with 10⁵ PFU of W7-791 or the same volume of PBS. One month after such immunization, they were inoculated with four times of the WSN virus from MLD₅₀. After the virus infection, the weight and survival conditions of the mice were regularly checked; (Panels D-E) each group of 5 mice intranasally immunized with 10⁵ PFU of W7-791 or the same volume of PBS. One month after such immunization, they were inoculated with four times of the PR8 virus from MLD₅₀. After the virus infection, the weight and survival conditions of the mice were regularly determined. *** represents a P-value <0.001.

In FIG. 7, a single immunization with W7-791 can activate powerful cross-protection against a lethal dose of heterosubtypic influenza infection.

In FIG. 7, (Panels A-B) 6 mice were immunized intranasally with 10⁶ PFU of W7-791 or PBS. Three weeks after immunization, the mice were inoculated with 2 MLD₅₀ Cam/H5. The body weight and survival conditions of the mice were checked at the indicated time points. (Panels C-D) One month after mice were vaccinated intranasally with 10⁵ PFU of W7-791 (n=9) or PBS (n=6), the mice were inoculated with 2 MLD₅₀ Vic/H3. The body weight and survival conditions of the mice were checked at the indicated time points. (Panels E-F) Three weeks after newborn mice were vaccinated with W7-791, they were inoculated with WSN (10⁵ or 10⁶ TCID₅₀) and HK68/H3 (10⁶ or 10⁷ TCID₅₀). Weight change in mice were observed. *** represents a P-value <0.001.

FIG. 8 W7-791 was able to better protect mice infected with the heterosubtypic H3 virus.

In FIG. 8, C57BL/6 mice were immunized with 10⁶ TCID₅₀ FluMist (2016) or W7-791. A month later, these mice were infected with 2 MLD₅₀ HK68 H3N1. Two figures respectively show changes in the body weight and survival of such mice after infection.

In FIG. 9, W7-791 was able to activate humoral and cell-mediated responses.

In FIG. 9, (Panel A) the virus titer of mouse lung homogenate was determined; (Panel B) an HAI assay was performed of the serum of immunized mice; (Panel C) a determination of anti-influenza virus antibody in serum of the immunized mice was made; (Panel D) a trace neutralizing experiment was performed of the neutralizing antibody titer in the serum of the mice immunized with W7-791; (Panels E-F) the serum of the mice immunized with W7-791 was adopted to non-immunized mice, which were inoculated with a lethal dosage of WSN HK68/H3 virus 24 hours later. The survival of such mice was observed and recorded at each time point; (Panels G-H) T-cells of mice immunized with W7-791 were adopted to non-immunized mice, which were inoculated with a lethal dosage of WSN HK68/H3 virus 24 hours later. The survival of such mice was observed and recorded at each time point.

In FIG. 10, a single immunization with W7-791 can activate a function of protection in the bodies of ferrets against subtypes of the viruses.

In FIG. 10, (Panel A) ferrets were inoculated intranasally with 10⁶, 10⁷ or 10⁸ TCID₅₀ W7-791 or PBS and their body temperature changes were observed. (Panel B) The ferrets were infected with W7-791 or 10⁶ TCID₅₀ WSN and were then clinically scored. (Panel C) An HAI analysis showed that the antibody titer in the serum of ferrets immunized with W7-791 increased. (Panel D) The HAI analysis showed that 21 days after the infection, H1HA or H3HA anti-antibody in their serum increased. After immunized or non-immunized ferrets were inoculated with 10⁶ TCID₅₀ WSN or HK68/H, a virus titer (Panel E,F) was evaluated and (Panel G,H) clinically scored.

### DETAILED DESCRIPTION OF THE INVENTION

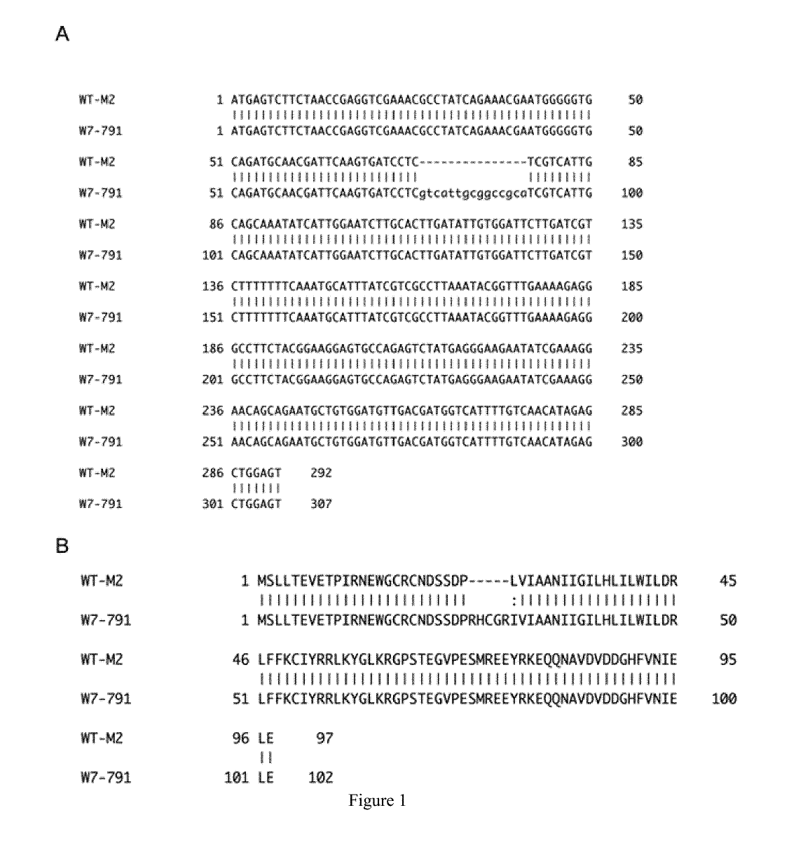

The present invention provides an influenza virus strain, whose depository number is CGMCC No. 13784 (W7-791). W7-791 has an insertional mutation (SEQ ID NO: 5) inserted after the first 78 nt of the M2 coding region of a wild-type influenza virus strain, as shown in FIG. 1.

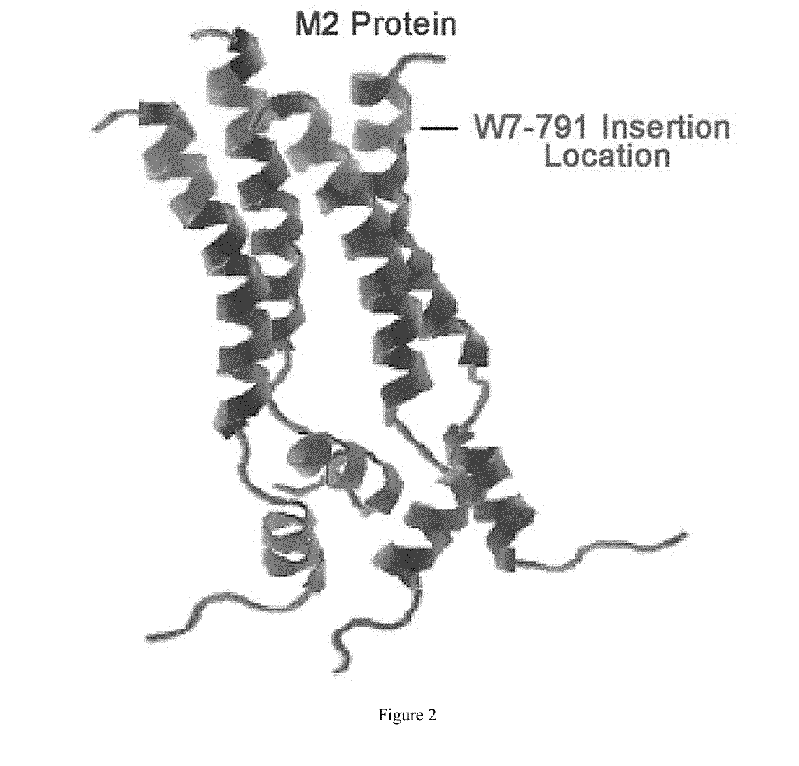

Segment 7 of the Influenza A virus genome encodes the M proteins, M1 and M2. M1 is a matrix protein and M2 forms an ion channel. Based on the wildtype and consensus M2 protein sequences, about the first 24 amino acid residues make up the ectodomain (ED) and the amino acids from about the 25-43 aa positions make up the transmembrane domain (TMD) of the ion channel. As shown in FIG. 1, the insertional mutation of W7-791 is located near the end (i.e., C-terminal end of the ectodomain) and the beginning (i.e., N-terminal end) of the transmembrane domain. As shown in FIG. 2, the insertional mutation is located at the cytoplasmic portion of the ion channel.

Like Influenza A virus, segment 7 of the Influenza B virus genome encodes the M1 matrix protein and an M2 protein that forms ion channel, which is commonly referred to as “BM2”. Based on wildtype and consensus sequences, the transmembrane domain of BM2 begins at about amino acid residues 5-8. Both the transmembrane domains of M2 and BM2 contain a HXXXW motif. Segment 6 of the Influenza C virus genome encodes the M1 matrix protein and an M2 protein that forms ion channel, which is commonly referred to as “CM2”. Based on various structural studies in the art, the transmembrane domain of CM2 begins at about amino acid residues 48-52. There are few studies on the Influenza D virus. However, it is believed that Influenza D virus contains an M2 protein that forms ion channel, “DM2”, that is like CM2, and as an ion channel, the DM2 protein has a transmembrane domain.

Therefore, in some embodiments, the present invention is directed to a recombinantly engineered influenza virus which comprises an insertional mutation in a genomic segment that encodes an M2 protein. In other words, the recombinantly engineered influenza viruses of the present invention contain a nucleic acid molecule that encodes a mutated M2 protein, wherein said mutated M2 protein comprises a given M2 protein sequence which has an exogenous sequence inserted therein.

In some embodiments, the genomic segment is segment 7 of a virus belonging to Influenza A virus. In some embodiments, the genomic segment is segment 7 of a virus belonging to Influenza B virus. In some embodiments, the genomic segment is segment 6 of a virus belonging to Influenza C virus. In some embodiments, the genomic segment is segment 6 of a virus belonging to Influenza D virus. In some embodiments, the M2 protein is a consensus or wildtype sequence of a M2 protein of Influenza A virus. In some embodiments, the M2 protein is a consensus or wildtype sequence of a BM2 protein of Influenza B virus. In some embodiments, the M2 protein is a consensus or wildtype sequence of a CM2 protein of Influenza C virus. In some embodiments, the M2 protein is a consensus or wildtype sequence of a DM2 protein of Influenza D virus. In some embodiments, the M2 protein is a protein of Influenza D virus that is similar in function and structure to a CM2 protein of Influenza C virus.

In some embodiments, the insertional mutation encodes an amino acid segment that comprises or consists of 4-6 amino acid residues of SEQ ID NO: 6. In some embodiments, the insertional mutation encodes an amino acid segment that comprises or consists of 4-6 contiguous amino acid residues of SEQ ID NO: 6. In some embodiments, the insertional mutation encodes an amino acid segment that comprises or consists of SEQ ID NO: 6. In some embodiments, the insertional mutation comprises or consists of SEQ ID NO: 7, wherein each “n” are independently any nucleotide. In some embodiments, the insertional mutation comprises or consists of SEQ ID NO: 5.

For convenience, when describing the location of the insertional mutation, reference will be made to the given M2 amino acid sequence. Additionally, it is noted that the actual positions of the amino acid residues that make up the ectodomain and the transmembrane domain can vary, e.g., by up to about 3 or 4 residues, and such depends on the given influenza virus. Nevertheless, one skilled in the art can readily determine end of the ectodomain and/or the beginning of the transmembrane domain of a given M2 protein using methods in the art, e.g., sequence alignment, protein modeling, and/or crystallography. In some embodiments, the insertional mutation is located at or near, e.g., within about 1-5 amino acid residues, of the C-terminal end of the ectodomain of the M2 protein. In some embodiments, the insertional mutation is located at or near, e.g., within about 1-5 amino acid residues, of the N-terminal end of the transmembrane domain of the M2 protein. In some embodiments, the insertional mutation is located at the cytoplasmic portion of the ion channel formed by the M2 protein. In some embodiments, the M2 protein contains a HXXXW motif. In some embodiments, the insertional mutation is located upstream of the HXXXW motif.

In some embodiments, the insertional mutation is located within about the 23^{rd} to 27^{th} amino acid residue of an M2 protein of a virus belonging to Influenza A virus. In some embodiments, the insertional mutation is located after the 25^{th} amino acid residue of the M2 protein of a virus belonging to Influenza A virus. In some embodiments, the insertional mutation is directly linked to the C-terminal end of the 25^{th} amino acid residue of the M2 protein of a virus belonging to Influenza A virus. In some embodiments, the insertional mutation is located after the 26^{th} amino acid residue of the M2 protein of a virus belonging to Influenza A virus. In some embodiments, the insertional mutation is directly linked to the C-terminal end of the 26^{th} amino acid residue of the M2 protein of a virus belonging to Influenza A virus. In some embodiments, the insertional mutation is located within about the 5^{th} to 8^{th} amino acid residue of a BM2 protein of a virus belonging to Influenza B virus. In some embodiments, the insertional mutation is located within about the 48^{th} to 52^{nd} amino acid residue of a CM2 protein of a virus belonging to Influenza C virus.

In some embodiments, the M2 protein is of a virus belonging to Influenza A virus, and the virus is an H1, H2, H3, H5, H6, H7, H9, or H10 subtype. In some embodiments, the M2 protein is of a virus belonging to Influenza A virus, and the virus is an H1, H2, or H3 subtype. In some embodiments, the M2 protein is of a virus belonging to Influenza A virus, and the virus is an N1, N2, N6, N7, N8, or N9 subtype. In some embodiments, the M2 protein is of a virus belonging to Influenza A virus, and the virus is an N1 or N2 subtype. In some embodiments, the M2 protein is of a virus belonging to Influenza A virus, and the virus is an H1, H2, H3, H5, H6, H7, H9, or H10 subtype and an N1, N2, N6, N7, N8, or N9 subtype. In some embodiments, the M2 protein is of a virus belonging to Influenza A virus, and the virus is an N1 or N2 subtype. In some embodiments, the M2 protein is of a virus belonging to Influenza A virus, and the virus is an H1, H2, or H3 subtype and an N1 or N2 subtype. In some embodiments, the M2 protein is of a virus belonging to Influenza A virus, and the virus is an H1N1, H2N1, H3N1, H5N1, H6N1, H7N1, H9N1, H10N1, H1N2, H2N2, H3N2, H5N2, H6N2, H7N2, H9N2, H10N2, H1N6, H2N6, H3N6, H5N6, H6N6, H7N6, H9N6, H10N6, H1N7, H2N7, H3N7, H5N7, H6N7, H7N7, H9N7, H10N7, H1N8, H2N8, H3N8, H5N8, H6N8, H7N8, H9N8, H10N8, H1N9, H2N9, H3N9, H5N9, H6N9, H7N9, H9N9, or H10N9 subtype. In some embodiments, the M2 protein is of a virus belonging to Influenza A virus, and the virus is an H1N1, H2N1, H3N1, H1N2, H2N2, or H3N2 subtype. In some embodiments, the M2 protein is of a virus belonging to Influenza A virus and of the H1N1 subtype.

In some embodiments, the M2 protein is of a virus belonging to Influenza A virus, and the virus is Influenza A virus A/WSN/1933. In some embodiments, the M2 protein is of a virus belonging to Influenza A virus, and the virus is Influenza A virus A/Puerto Rico/8 H1N1 (PR8).

In some embodiments, the M2 protein comprises or consists of an amino acid sequence having at least 85%, preferably at least 90%, more preferably at least 95%, and most preferably at least 99% sequence identity to

### MSLLTEVETPIRNEWGCRCNXSSDPXXIAANIIGILHXXXWILDRLFFKCIYRRXKYGLKXGPSTE GVPXSMREEYRKEQQXAVDXDDGHFVXIEXX (SEQ ID NO: 12)

wherein each X are independently any amino acid residue. In some embodiments, the M2 protein comprises or consists of an amino acid sequence having at least 85%, preferably at least 90%, more preferably at least 95%, and most preferably at least 99% sequence identity to SEQ ID NO: 2.

In some embodiments, the recombinantly engineered influenza virus comprises a genomic segment that encodes a mutated M2 protein that comprises or consists of an amino acid sequence having at least 85%, preferably at least 90%, more preferably at least 95%, and most preferably at least 99% sequence identity to SEQ ID NO: 4.

In some embodiments, the recombinantly engineered influenza virus is W7-791.

In some embodiments, the present invention is directed to a composition which comprises, consists essentially of, or consists of one or more recombinantly engineered influenza viruses as disclosed herein, e.g., according to any one of paragraphs [0045] to

In some embodiments, the composition further comprises a pharmaceutically acceptable carrier and/or an adjuvant.

As used herein, a “recombinantly engineered influenza virus” refers to a virus that has been engineered to contain an insertional mutation in its M2 protein as described herein, e.g., paragraphs [0045] to [0054]. One or more recombinantly engineered influenza viruses as described herein may be used to vaccinate a subject against influenza.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., J. Mol. Biol. 215:403-410 (1990). Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

In some embodiments, the present invention is directed to mutated M2 proteins and compositions thereof. As used herein, a “mutated M2 protein” refers to an M2 protein of an influenza virus that contains an insertional mutation as disclosed herein.

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus.

Mutated M2 proteins of the present invention may be made using methods known in the art including chemical synthesis, biosynthesis or in vitro synthesis using recombinant DNA methods, and solid phase synthesis. See e.g., Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, Ill., which are herein incorporated by reference. Mutated M2 proteins of the present invention may be purified using protein purification techniques known in the art such as reverse phase high-performance liquid chromatography (HPLC), ion-exchange or immunoaffinity chromatography, filtration or size exclusion, or electrophoresis. See Olsnes and Pihl (1973) Biochem. 12(16):3121-3126; and Scopes (1982) Protein Purification, Springer-Verlag, N.Y., which are herein incorporated by reference. Alternatively, polypeptides of the present invention may be made by recombinant DNA techniques known in the art. Thus, polynucleotides that encode the mutated M2 proteins of the present invention are contemplated herein. In some embodiments, the polypeptides and polynucleotides of the present invention are isolated.

As used herein, an “isolated” compound refers to a compound that is isolated from its native environment. For example, an isolated polynucleotide is a one which does not have the bases normally flanking the 5′ end and/or the 3′ end of the polynucleotide as it is found in nature. As another example, an isolated polypeptide is a one which does not have its native amino acids, which correspond to the full-length polypeptide, flanking the N-terminus, C-terminus, or both.

In some embodiments, the recombinantly engineered influenza viruses and mutated M2 proteins of the present invention are substantially purified. As used herein, a “substantially purified” compound refers to a compound that is removed from its natural environment and/or is at least about 60% free, preferably about 75% free, and more preferably about 90% free, and most preferably about 95-100% free from other macromolecular components or compounds with which the compound is associated with in nature or from its synthesis.

In some embodiments, the present invention provides antibodies against one or more mutated M2 proteins. As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)2, disulfide linked Fvs (sdFvs), Fvs, and fragments comprising or alternatively consisting of, either a VL or a VH domain.

In some embodiments, antibodies of the present invention specifically bind one or more mutated M2 proteins as described herein. In some embodiments, the antibodies are raised against a mutated M2 protein as described herein. In some embodiments, the antibodies specifically bind a recombinantly engineered influenza virus as described herein. In some embodiments, the antibodies are monoclonal antibodies. In some embodiments, the monoclonal antibodies are obtained from rabbit-based hybridomas. As used herein, a compound (e.g., receptor or antibody) “specifically binds” a given target (e.g., ligand) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure. An example of an antibody that specifically binds a recombinantly engineered influenza virus is an antibody that binds the recombinantly engineered influenza virus with greater affinity, avidity, more readily, and/or with greater duration than it does to other compounds.

As used herein, “binding affinity” refers to the propensity of a compound to associate with (or alternatively dissociate from) a given target and may be expressed in terms of its dissociation constant, Kd. In some embodiments, an antibody according to the present invention has a Kd of 10⁻⁵ or less, 10⁻⁶ or less, preferably 10⁻⁷ or less, more preferably 10⁻⁸ or less, even more preferably 10⁻⁹ or less, and most preferably 10⁻¹⁰ or less. Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of the antibody for the given target is at least 1-fold or more, preferably at least 5-fold or more, more preferably at least 10-fold or more, and most preferably at least 100-fold or more than its binding affinity for the specified alternative.

Compositions of the present invention, including pharmaceutical compositions and vaccines, include one or more recombinantly engineered influenza viruses and/or one or more mutated M2 proteins as disclosed herein.

As used herein, the phrase “consists essentially of” in the context of a composition containing one or more recombinantly engineered influenza viruses or means that the composition may comprise one or more supplementary agents, binders, adjuvants, adsorption delaying agents, antibacterial agents, antifoaming agents, antifungal agents, antioxidants, buffering agents, diluents, disintegration agents, dispersing agents, emulsifying agents, erosion facilitators, filling agents, flavoring agents, lubricants, pH adjusting agents, pharmaceutically acceptable carriers, plasticizers, preservatives, solubilizers, stabilizers, surfactants, suspending agents, thickening agents, viscosity enhancing agents, wetting agents, and the like, and so long as the additional ingredients do not interfere with the activity of the one or more recombinantly engineered influenza viruses. A composition that consists of one or more recombinantly engineered influenza viruses is one which comprises the one or more recombinantly engineered influenza viruses as the sole active ingredient, i.e., the composition does not contain any supplementary agents, but may include ingredients typically used in pharmaceutical compositions, e.g., binders, adjuvants, adsorption delaying agents, antibacterial agents, antifoaming agents, antifungal agents, antioxidants, buffering agents, diluents, disintegration agents, dispersing agents, emulsifying agents, erosion facilitators, filling agents, flavoring agents, lubricants, pH adjusting agents, pharmaceutically acceptable carriers, plasticizers, preservatives, solubilizers, stabilizers, surfactants, suspending agents, thickening agents, viscosity enhancing agents, wetting agents, and the like.

The term “pharmaceutical composition” refers to a composition suitable for pharmaceutical use in a subject. A pharmaceutical composition generally comprises an effective amount of an active agent, e.g., one or more recombinantly engineered influenza viruses according to the present invention, and a pharmaceutically acceptable carrier. The term “effective amount” refers to a dosage or amount sufficient to produce a desired result. The desired result may comprise an objective or subjective improvement in the recipient of the dosage or amount, e.g., long-term survival, effective prevention of a disease state, and the like.

One or more recombinantly engineered influenza viruses according to the present invention may be administered, preferably in the form of pharmaceutical compositions, to a subject. Preferably the subject is mammalian, more preferably, the subject is human. Preferred pharmaceutical compositions are those comprising at least one recombinantly engineered influenza virus in a therapeutically effective amount or an immunogenic amount, and a pharmaceutically acceptable vehicle.

Vaccines according to the present invention provide a protective immune response when administered to a subject. As used herein, a “vaccine” according to the present invention is a pharmaceutical composition that comprises an immunogenic amount of at least one recombinantly engineered influenza virus and provides a protective immune response when administered to a subject. The protective immune response may be complete or partial, e.g., a reduction in symptoms as compared with an unvaccinated subject.

As used herein, an “immunogenic amount” is an amount that is sufficient to elicit an immune response in a subject and depends on a variety of factors such as the immunogenicity of the given recombinantly engineered influenza virus, the manner of administration, the general state of health of the subject, and the like. The typical immunogenic amounts for initial and boosting immunizations for therapeutic or prophylactic administration may range from about 120 μg to 8 mg per kilogram of body weight of a subject. For example, the typical immunogenic amount for initial and boosting immunization for therapeutic or prophylactic administration for a human subject of 70 kg body weight ranges from about 8.4 mg to about 560 mg, preferably about 10-100 mg, more preferably about 10-20 mg, per about 65-70 kg body weight of a subject. Examples of suitable immunization protocols include an initial immunization injection (time 0), followed by booster injections at 4, and/or 8 weeks, which these initial immunization injections may be followed by further booster injections at 1 or 2 years if needed.

As used herein, a “therapeutically effective amount” refers to an amount that may be used to treat, prevent, or inhibit a given disease or condition, such as influenza, in a subject as compared to a control. Again, the skilled artisan will appreciate that certain factors may influence the amount required to effectively treat a subject, including the degree of exposure to influenza, previous treatments, the general health and age of the subject, and the like. Nevertheless, therapeutically effective amounts may be readily determined by methods in the art. It should be noted that treatment of a subject with a therapeutically effective amount or an immunogenic amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

The compositions of the present invention may include an adjuvant. As used herein, an “adjuvant” refers to any substance which, when administered in conjunction with (e.g., before, during, or after) a pharmaceutically active agent, such as a recombinantly engineered influenza virus according to the present invention, aids the pharmaceutically active agent in its mechanism of action. Thus, an adjuvant in a vaccine according to the present invention is a substance that aids the at least one recombinantly engineered influenza virus in eliciting an immune response. Suitable adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, nor-MDP), N-acetylmuramyl-Lalanyl-D-isoglutaminyl-L-alanine-2-(1′-2′-dipa-lmitoyl-sn-glycero-3-hydroxyphosphoryloxy)-ethylamine (CGP 19835A, MTP-PE), and RIBI, which comprise three components extracted from bacteria, monophosphoryl lipid A, trehalose dimycolate and cell wall skeleton (NPL+TDM+CWS) in a 2% squalene/Tween 80 emulsion. The effectiveness of an adjuvant may be determined by methods in the art.

Pharmaceutical compositions of the present invention may be formulated for the intended route of delivery, including intravenous, intramuscular, intraperitoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations of the present invention may be optimized for increased stability and efficacy using methods in the art. See, e.g., Carra et al. (2007) Vaccine 25:4149-4158, which is herein incorporated by reference.

The compositions of the present invention may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular recombinantly engineered influenza virus used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., REMINGTON: THE SCIENCE AND PRACTICE OF PHARMACY. 20^{th} ed. (2000) Lippincott Williams & Wilkins. Baltimore, Md., which is herein incorporated by reference.

The pharmaceutical compositions of the present invention may be provided in dosage unit forms. As used herein, a “dosage unit form” refers to physically discrete units suited as unitary dosages for the subject to be treated; each unit containing a predetermined quantity of the one or more recombinantly engineered influenza virus calculated to produce the desired therapeutic effect in association with the required pharmaceutically acceptable carrier. The specification for the dosage unit forms of the invention are dictated by and directly dependent on the unique characteristics of the given recombinantly engineered influenza virus and desired therapeutic effect to be achieved, and the limitations inherent in the art of compounding such an active compound for the treatment of individuals.

Toxicity and therapeutic efficacy of recombinantly engineered influenza viruses according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Recombinantly engineered influenza viruses which exhibit large therapeutic indices are preferred. While recombinantly engineered influenza viruses that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more recombinantly engineered influenza viruses according to the present invention can be estimated initially from cell culture assays. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

In some embodiments, the present invention is directed to kits which comprise one or more recombinantly engineered influenza viruses, optionally in a composition, packaged together with one or more reagents or drug delivery devices for preventing, inhibiting, reducing, or treating influenza in a subject. Such kits include a carrier, package, or container that may be compartmentalized to receive one or more containers, such as vials, tubes, and the like. In some embodiments, the kits optionally include an identifying description or label or instructions relating to its use. In some embodiments, the kits comprise the one or more recombinantly engineered influenza viruses, optionally in one or more unit dosage forms, packaged together as a pack and/or in drug delivery device, e.g., a pre-filled syringe. In some embodiments, the kits include information prescribed by a governmental agency that regulates the manufacture, use, or sale of compounds and compositions according to the present invention.

In some embodiments, the present invention provides a kit comprising one or more recombinantly engineered influenza viruses or compositions thereof packaged together. In some embodiments, one or more recombinantly engineered influenza viruses are packaged together with one or more supplementary agents. One or more components of a kit according to the present invention can be enclosed within an individual container. In some embodiments, the kits comprise the one or more recombinantly engineered influenza viruses, optionally in one or more unit dosage forms, packaged together as a pack and/or in drug delivery device, e.g., a pre-filled syringe. In some embodiments, the kits comprise one or more recombinantly engineered influenza viruses packaged together with a device for intranasal or intravenous administration. In some embodiments, the kits comprise one or more recombinantly engineered influenza viruses packaged together with an umbilical venous catheter. In some embodiments, the kits include a carrier, package, or container that may be compartmentalized to receive one or more containers, such as vials, tubes, and the like.

### EXAMPLES

The following examples are intended to illustrate but not to limit the invention.

### Cell Culture

HEK293T cells were cultured in DMEM supplemented with 5% heat-inactivated fetal bovine serum (FBS). MDCK cells were maintained in DMEM containing 5% FBS, penicillin/streptomycin (100 U/mL and 50 μg/mL, respectively), and 1 mM sodium pyruvate at 37° C. with 5% CO₂.

### Example 1

### Generation of M Gene Segment Mutant Plasmid Library and Functional Profiling

To create the mutant plasmid library of the M gene segment of influenza A virus A/WSN/1933, a 15 nt insert (5′-NNNNNTGCGGCCGCA-3′ (SEQ ID NO: 7)), wherein N=duplicated 5 nucleotides from target DNA, was randomly inserted by Mu-transposon-mediated mutagenesis (MGS kit, Finnzymes) according to the manufacturer's instructions. The M gene mixed mutant pool was transformed into E. coli DH10B by electroporation at 2.0 kV, 200 Ω, 25 μF (ElectroMax DH10B, Invitrogen). The mutant M gene plasmid and seven remaining WT plasmids were transfected concomitantly into HEK293T cells for virus generation. Three days after transfection, the supernatant was collected and transferred to MDCK cells for propagation. Virus was collected after 48 hr, then either stored or used for further propagation for up to four passages. RNA was isolated with the TRIzol reagent (Invitrogen) after each generation. RT-PCR was carried out with the iScript cDNA Synthesis kit (Bio-Rad) to create cDNA. Three gene-specific forward primers approximately 400 bp apart in the M-gene segment (5′-AGCAAAAGCAGGTAGATATT-3′ (SEQ ID NO: 8), 5′-GGGGCCAAAGAAATAGCACT-3′ (SEQ ID NO: 9), and 5′-TCCTAGCTCCAGTGCTGGTC-3′ (SEQ ID NO: 10)) and a Vic-labeled insertion-specific mini-primer (5′-TGCGGCCGCA-3′ (SEQ ID NO: 11)) were used to amplify fragments containing the 15 nt insert using KOD Hot-Start polymerase (Novagen). The PCR conditions were set to 95° C. for 10 min (1 cycle); 95° C. for 45 s, 52° C. for 30 s, and 72° C. for 90 s (30 cycles); and 72° C. for 10 min (1 cycle). The fluorescent-labeled PCR products were analyzed in duplicate with a Liz-500 size standard (Applied Biosystems) using a 96-capillary genotyper (3730×1 DNA Analyzer, Applied Biosystems) at the UCLA GenoSeq Core facility. Sequencing data were analyzed for clarity using ABI software, with the following criteria: (1) all data passed the standard default detection level; (2) the first 70 bp were removed due to non-specific background noise; (3) all data were aligned to the nearest base pair in the influenza A WSN matrix gene; and (4) all genotyping experimental data were normalized with WT WSN-infected cells, non-transfected cells, and a different gene library as controls. This eliminated non-specific data from the PCR, primers, and the DNA Analyzer. For infection in vivo, the mutant virus pool was titered, concentrated by ultra-centrifugation and re-titered, and used for mouse injection. Two dpi, the lungs were harvested, homogenized, and resuspended in TRIzol for RNA isolation, followed by the same procedures as described above. PBS or WSN-infected mice served as controls.

### Virus Strains

We used the influenza A/WSN/1933 reverse genetics system to generate seasonal A/H1N1 virus (Hoffmann et al.,). This strain is a mouse-adapted influenza virus and has been used as the parental strain to generate potential LAIVs using transposon mutagenesis. The eight plasmids containing the cDNA of A/WSN/33 (gift from Dr. Yuying Liang at Emory University) were transfected into HEK293T cells using TransIT LT-1 (Panvera) by the manufacturer's protocol. The virus was serially passaged three times in MDCK cells to a final titer of 10^{7.4} PFU/mL. Influenza virus A/Puerto Rico/8/1934 (seasonal A/H1N1 virus) was a gift from Dr. Yuying Liang. The virus was serially passaged three times in MDCK cells to a titer of 10^{7.5} PFU/mL. The MLD₅₀ of both strains was determined in C57BL/6 mice.

Influenza virus A/Victoria/3/75 (seasonal A/H3N2 virus), A/Wisconsin/65/05 (seasonal A/H3N2 virus), and A/Hongkong/68 (seasonal A/H3N1 virus) were gifts from Dr. Ioanna Skountzou at Emory University. These viruses were amplified using MDCK cells for two to three passages to a final titer of 10^{5.5} PFU/mL, 10^{5.4} PFU/mL, and 10⁷ PFU/mL, respectively. The MLD₅₀ was determined in C57BL/6 and BALB/c mice.

Influenza virus A/Cambodia/P0322095/05 (highly pathogenic avian influenza H5N1 virus) was originally isolated from human patients at the Pasteur Institute in Cambodia (Buchy et al.,). Virus was propagated in MDCK cells and virus-containing supernatants were pooled, clarified by centrifugation, and stored at −80° C. The TCID₅₀ and the MLD₅₀ of the viruses were determined in MDCK cells and in BALB/c mice, respectively, and were calculated as described previously (Ding et al.,).

### Virus Titrations

The concentration of infectious viruses was determined by plaque assay and end-point titrations. Plaque assays were performed in MDCK cells and calculated as PFU/μL of supernatant. The viral samples were serially diluted in dilution buffer (PBS with 10% BSA, CaCl₂, 1% DEAE-dextran, and MgCl₂). Diluents were added to a monolayer of MDCK cells in 6-well plates for 1 hr at 37° C., and then covered with growth medium containing 1% low-melting agarose and TPCK-treated trypsin (0.7 μg/mL). Infected cells were stained after 48 hr (1% crystal violet, 20% ethanol, in PBS) to visualize the plaques. Virus titrations were performed by end-point titration in MDCK cells. MDCK cells were inoculated with 10-fold serial dilutions of the virus, then washed with PBS once 1 hr after inoculation, and cultured in DMEM for 48 hr to visualize cell viability. The viral titer was determined by luminescence assay or by plaque assay. To measure the growth of individual mutants in vitro, an influenza virus-responsive Gaussia luciferase (gLuc) reporter system was used. Briefly, the gLuc coding region was inserted in the reverse-sense orientation between a human RNA polymerase I promoter and a murine RNA polymerase I terminator. The gLuc coding sequence was flanked by the UTRs from the PA segment of influenza virus A/WSN/33 strain so that gLuc expression is dependent on influenza virus infection. The gLuc reporter was transfected into HEK293Ts for 24 hr before the supernatants containing mutant or WT influenza viruses were added. Upon active infection, gLuc is released into the supernatant and can be quantified with Renilla luciferase substrate (Promega).

### Animals

### Adult Mice

Female C57BL/6 mice, 6-8 weeks old, were purchased from the Jackson Laboratory. All animals were housed in pathogen-free conditions within the UCLA animal facilities.

### Neonatal Mice

Fifteen-day-old BALB/c mice (Vital River Beijing) weighing 6-9 g were inoculated i.n. with PBS, 10⁴ TCID₅₀ of WSN virus, or dilutions of W7-791. For the dose-dependent experiment, mice were inoculated i.n. with 10⁶, 10⁷, and 10⁸ TCID₅₀ of W7-791. Sixteen days post-treatment, mice were challenged i.n. with a lethal dose (10⁵ or 10⁶ TCID₅₀/mouse) of WSN or (10⁶ or 10⁷ TCID₅₀/mouse) A/Hong Kong/68 H3N1 (HK68/H3) in a 30 μL volume. Randomly selected mice from each group were sacrificed for pathological examinations of the lung at 4 and 6 dpi. Then the lungs were homogenized to measure viral titer using end-point-dilution assays.

### Ferrets

Healthy young adult outbred female ferrets (Mustela putorius furo; between 4 and 5 months of age) were purchased from a commercial breeder (Wuxi) and confirmed to be seronegative by HAI assay to A/WSN/1933 (H1N1), A/Victoria/3/75 (H3N2), HK68 (H3N1), and W7-791 (H1N1). A minimum of three independently housed ferrets were inoculated i.n. with 0.5 mL (0.25 mL per nostril) of 10⁶, 10⁷, or 10⁸ TCID₅₀ of W7-791 or PBS. Anesthesia was performed on the quadriceps muscles of the left hind leg with a total volume of 0.02 mL Lumianning (Hua Mu Animal Care). Serum samples were collected at days 0, 7, 14, 21, and 28 post-immunization for HAI studies. Nasal washes were collected 0-7 days after immunization. Four weeks after immunization, the ferrets were challenged i.n. with 10⁶ TCID₅₀ of WSN (H1N1) or HK68 (H3N1). Weights and temperatures were monitored daily for 7 days after inoculation. Nasal washes were collected 0-7 days after the challenge. Clinical signs were evaluated 3 days prior to vaccination, then 9, 11, 13, and 15 dpi, and 2 days prior to challenge and 1-7 dpi. The clinical signs were scored as previously described (Reuman et al.,). All animal studies were performed according to the guidelines of the UCLA Animal Research Committee.

### Mouse Immunization and Challenge

Female C57BL/6 and BALB/c mice were randomly divided into groups of five or six mice. Groups were inoculated i.n. or intratracheally with either PBS or W7-791 in a volume of 50 μL. Intratracheal injection was performed by anesthetizing mice intraperitoneally with a ketamine/xylazine mixture, then surgically exposing the trachea for direct injection of 30 μL of solution with a sterile 27G needle (Shahangian et al.,). Four weeks after immunization, all mice were challenged i.n. or intratracheally with an influenza strain in a 50 μL volume: A/WSN/1933 (H1N1) at 4 MLD₅₀, A/Puerto Rico/8/1934 (H1N1) at 4 MLD₅₀, A/Cambodia/P0322095/05 (HPAI-H5N1) at 2 MLD₅₀, or A/Victoria/3/75 (H3N2) at 2 MLD₅₀. Mice were monitored and recorded daily for signs of illness, such as lethargy, ruffled hair, and weight loss. When mice lost 30% or more of their original weight, they were euthanized and counted as dead. For the adoptive transfer experiment, female C57BL/6 mice were randomly divided into two sets of vaccinated or unvaccinated groups. Unvaccinated mice were sham immunized, whereas the vaccinated group received a single dose of W7-791 at 10⁶ PFU/mouse. One set from each group was used to harvest cells for the transfer experiment 4 weeks post-vaccination, while the other set was used as a vaccinated, but not transferred, control. Total CD4+ and CD8+ T cells were isolated from the spleens of the vaccinated and the unvaccinated mice using the Mouse Pan T Cell Isolation Kit and MS columns (Miltenyi Biotec). On the same day, the cells from the same group were pooled, and about 10^{6.3} T cells/mouse were injected via the retro-orbital route to a new set of naive female C57BL/6 mice. Likewise, sera were isolated from either the vaccinated or unvaccinated groups and matching groups were pooled, then 100 μL/mouse of serum was administered retro-orbitally to a new set of naive female C57BL/6 mice. The mice in all groups were challenged i.n. at 24 hr post-adoptive transfer with 2 MLD₅₀ of WSN or 2 MLD₅₀ of HK68/H3.

### In Vivo Challenge Using HPAI Virus H5N1

All animal protocols were approved by the Institutional Animal Care and Use Committee at the Pasteur Institute of Cambodia. Female BALB/c mice (Mus musculus) at the age of 6-8 weeks were purchased from Charles River Laboratories and housed in microisolator cages ventilated under negative pressure with HEPA-filtered air and a 12/12 hr light/dark cycle. Virus challenge studies were conducted in BSL3 facilities at the Pasteur Institute of Cambodia. Before each inoculation or euthanasia procedure, the mice were anesthetized by intraperitoneal (i.p.) injection of pentobarbital sodium (75 mg/kg; Sigma).

### Ethical Statement

All animal experiments were carried out at biosafety level 3 (BSL3) containment facilities complying with the Ethics Committee regulations of the Institut Pasteur, in accordance with EC directive 86/609/CEE and were approved by the Animal Ethics Committee of the Institut Pasteur in Cambodia (permit number VD100820). Before each inoculation or euthanasia procedure, the mice were anesthetized by i.p. injection of pentobarbital sodium, and all efforts were made to minimize suffering.

### Lung Homogenization

After animals were sacrificed, lungs were perfused by injecting 1 mL PBS containing 5 mM EDTA into the right ventricle. Whole lungs were removed and the lymph nodes were dissected away. The lungs were homogenized with 1 mL PBS containing a proteinase inhibitor cocktail (Roche Applied Science), and virus titers in lungs were evaluated by plaque assay. After homogenates were centrifuged at 10,000×g for 10 min, the supernatant was collected for genotyping.

### Structure Analysis

Conserved and viable mutations in the M gene were mapped onto the crystal structure of the monomeric M1 gene (PDB: 2Z16) and the tetrameric M2 gene (PDB:2L0J), which were obtained from PDB. The structure labeling was performed using PyMOL v.1.0.

### In Vitro Assays

### Cell Viability Assay

Cell viability was measured by CytoTox 96 Non-Radioactive Cytotoxicity Assay (Promega) according to the manufacturer's instructions.

### HAI Assay

Viruses A/WSN/1933, A/Puerto Rico/8/1934, A/Wisconsin/65/05, and A/Hong Kong/68 were diluted to 4 HA units and incubated with an equal volume of serially diluted sera for 30 min at room temperature. An equal volume of 1% chicken red blood cells was added to the wells and incubation continued on a gently rocking plate for 30 min at room temperature. Button formation was scored as evidence of HAI. Assays were performed in triplicate.

### Microneutralization Assay

MDCK cells (5×10⁵ cells per well) were seeded onto a 12-well culture plate in complete DMEM overnight. To test the neutralization activity of immune sera, serial 3-fold dilutions of sera were incubated with 10^{6.5} PFU/mL, 10^{4.4} PFU/mL, and 10^{4.2} PFU/mL of viruses A/WSN/1933, A/Hongkong/68, and A/Puerto Rico/8/1934 at the final volume of 100 μL at room temperature for 1 hr. After the incubation, the mixture was added onto a monolayer of MDCK cells and was incubated for 1 hr at 37° C. and then covered with growth medium containing 1% low-melting-point agarose and TPCK-treated trypsin (0.7 μg/mL). Infected cells were stained after 48 hr (1% crystal violet, 20% ethanol, in PBS) to visualize the plaques. Assays were performed in triplicate.

### Pseudovirus Neutralization Assay

H5N1 pseudotype virus expressing the H5HA derived from A/Cambodia/P0322095/05 (GenBank: ADM95463), the NINA (GenBank: AY555151) derived from A/Thailand/1(KAN-1)/2004, and a luciferase reporter gene were used in this experiment. The ferret sera were diluted in 2-fold serial dilutions from 1/20 to 1/1,280 and the mouse sera were diluted from 1/10 to 1/1,280. Sera from mice immunized by injection of H5HA DNA (GenBank: AAS65615) from A/Thailand/1(KAN-1)/2004 were used as a positive control. IC₅₀ values were defined as the dilution of a given immune serum that resulted in 50% reduction of RLA. The assay was performed in triplicate.

### Example 2

### W7-791

W7-791 was a live attenuated influenza virus vaccine strain obtained from the library of influenza virus mutants with hypermutations of the M gene all viruses by combining the emerging second generation high-throughput sequencing techniques with in vivo vaccine screening techniques. An analysis of specific viral genetic materials (viral genomic RNA) showed that W7-791 had the insertional mutation, GTCATTGCGGCCGCA (SEQ ID NO: 5) after the 78^{th} nt (referring to the corresponding cDNA of the virus genome) in its M2 gene region. Corresponding to the protein level, RHCGRI (SEQ ID NO: 6) peptide segment was inserted after the 26^{th} amino acid of the M2 protein of W7-791 virus. From the point of view of the overall structure of the M2 protein, this inserted peptide position is the cytoplasm segment located in the M2 protein ion channel (as shown in FIG. 1 and FIG. 2).

### Example 3

### Replication Kinetics W7-791 in vitro Cell Culture and in Mice

(1) Replication of W7-791 in cell culture

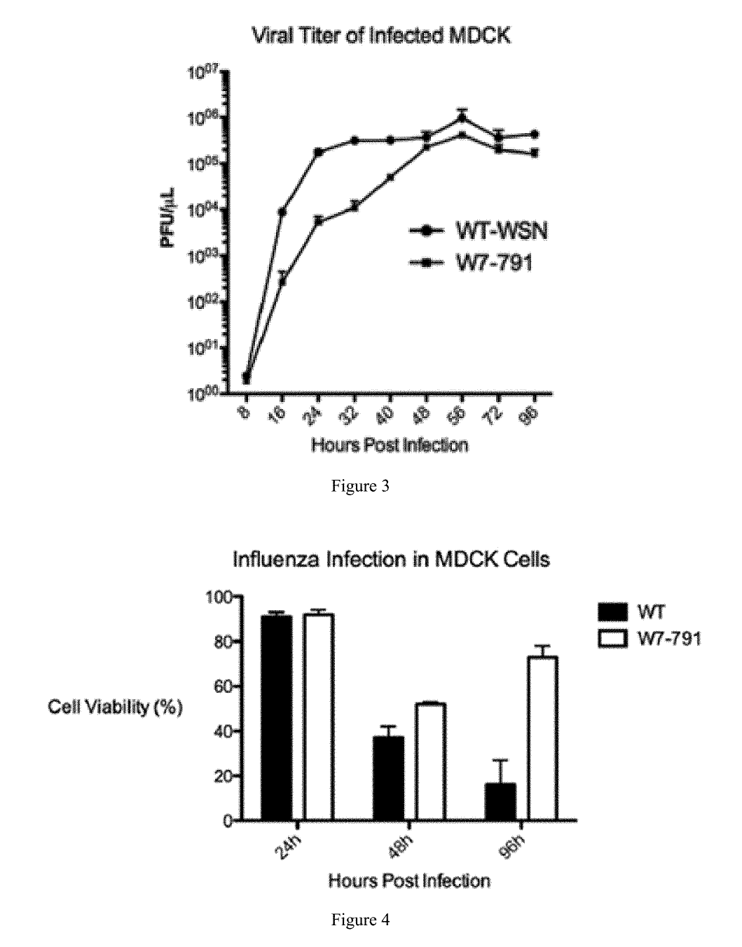

We used the wild-type WSN (WT—WSN) and to W7-791 MOI to infect MDCK cells at 0.25 and determined the virus titer of infected cell supernatants at different time points. The results showed that, although W7-791 replication was slower than that of WT-WSN, it showed good replicability in MDCK cells. At the peak point of replication, W7-791 could achieve substantially the same virus titer as WT-WSN virus titers (as shown in FIG. 3).

(2) Replication of W7-791 in mice

Although W7-791 could effectively replicate in mice in the first six days after infecting them and a higher virus titer could be detected in the lungs, at 6-8 days after the infection, it had been cleared from the body. At this time, almost no presence of such viruses could be detected. But throughout the course of infection, the mice did not present any flu-related symptoms.

### Example 4

### Safety and Genetic Stability of W7-791

A good attenuated live vaccine is required to be absolutely safe and its phenotype and genotype are required to be genetically stable between generations. Therefore, we performed a systematic and comprehensive evaluation of the safety and genetic stability of the attenuated vaccine candidate strain of W7-791. (1) A cell toxicity assay of W7-791 infection: We examined the cell viability of MDCK cells infected with W7-791 at different time points and found the cytotoxicity of W7-791 is significantly less than that of the WT-WSN virus (FIG. 4); (2) genetic stability test of the attenuated vaccine: to ensure that the vaccine will not experience reverse mutation and there will not be an atavistic phenomenon of the attenuated vaccine, we carried out a series of passages of the W7-791 virus in MDCK cells and mice and determined the gene sequence of the virus obtained from cells or mouse lung homogenate, especially the sequence of the M genes. We found that the mutation of the M genes of the W7-791 virus can be steadily and genetically inherited and that the occurrence of the phenomena of the insertion of deletion of mutation or reverse mutation would not be likely. In addition, with an increase in the number of passages, the W7-791 virus titers decreased gradually. This indicated that the mutations and genetic phenotypes carried by the W7-791 virus can be steadily passed on genetically.

In mice of 6-8 weeks of age that were vaccinated with the W7-791 virus with different titers, even when the amount of virus inoculated per mouse was as high as 10⁷ TCID₅₀, we did not find any weight loss and flu symptoms experienced by such mice. By comparison, mice infected with 10³ TCID₅₀ wild-type of virus experienced significant flu symptoms and weight losses. The viral load of mice six days after their infection with W7-791 was 100-fold lower than the virus titer in the lungs of mice infected with the wild type WSN virus and H3 subtype virus (FIG. 5, Panels A, B, C, and D). If the lungs of mice were observed four days after the infection, we found no significant lesions in the PBS group and the lungs of mice infected with W7-791, whereas mice infected with wild-type WSN virus showed severe lung tissue damage. To further confirm the safety of W7-791, we intranasally inoculated 15-day-old neonatal BALB/c mice with various amounts (10⁶, 10⁷ or 10⁸ TCID₅₀) of W7-791 or 10⁴ TCID₅₀ wild-type WSN virus. The results of mouse weight and lung lesion detection showed that no reduction in weight and lung lesions in rats inoculated with W7-791 were observed like those observed in mice infected with wild-type WSN virus (FIG. 5, Panel E). These results showed that mutated influenza virus W7-791 that we obtained by screening could only be replicated on a limited basis in vitro and in vivo and was a new attenuated virus strain very safe for both adult and neonatal mice.

### Example 5

### Immuno-Protective Capability of W7-791

(1) One immunization can effectively protect mice against an infection with a lethal dose of homosubtypes of influenza virus

Mice were immunized with W7-791. One month after immunization, mice were infected with 4 times of MLD₅₀ parent or wild-type WSN virus or homosubtype of PR8 virus. We did not find that mice from the group not immunized experienced any weight reduction and deaths during the experiment process, whereas mice immunized with W7-791 had always maintained a normal weight and additionally, did not present any flu symptoms (as shown in FIG. 6, Panels A-E). This indicated that one immunization with W7-791 would effectively protect mice against an infection with a lethal dose of homosubtypes of influenza virus.

(2) One immunization can effectively cross protect mice against infections by different subtypes of influenza virus

Since influenza viruses can be divided into different subtypes, there is a lack of cross protection among the subtypes of viruses. Traditional inactivated vaccines need to be constantly updated according to different subtypes of the versus that are epidemic in different time periods. Therefore, we further investigated whether W7-791 can provide the body with cross protection capabilities against infections by different subtypes of influenza viruses. For this purpose, we first used W7-791 with a dosage of 10⁶ pfu to immunize mice. Three weeks after the immunization, we used 2 MLD₅₀ amount of H5N1 subtype of highly pathogenic avian influenza virus A/Cambodia/P0322095/05 (Cam/H5) to challenge the immunized group and control group of mice. The results showed that non-immunized mice experienced various flu symptoms, severe weight losses and deaths; the immunized mice did not appear to experience any significant weight loss and exhibited good resistance to Cam/H5 (FIG. 7, Panels A and B). In addition, we also tested the immuno-protective function of W7-791 against A/Victoria/3/75 H3N2 (Vic/H3) of a phylogroup of influenza virus. Four weeks after mice were immunized with 10⁵ pfu of W7-791, they were infected with 2 MLD₅₀ of Vic/H3. The results showed that the weight of mice immunized with W7-791 only decreased by about 10% 3-5 days after the challenge and gradually returned to normal, whereas mice in the immunized control group had died off (FIG. 7, Panels C and D). In addition, we also examined whether W7-791 could provide cross protection for neonatal mice, thus being able to protect against a lethal dose of parental WSN virus or a lethal dose of subtypes of influenza viruses. 10⁶ TCID₅₀ of the W7-791 virus was used to immunize 15-day-old BALB/c mice, and then a lethal dose of the WSN virus (10⁵ or 10⁶ TCID₅₀/mice) or A/Hong Kong/68 H3N1 (HK68/H3) (10⁶ or 10⁷ TCID₅₀/mice) virus was used to challenge the mice. Similar to adult mice, all immunized mice received protection and cleared the virus from their bodies (FIG. 7, Panels E and F).

Finally, we compared the immunization effect of W7-791 with that of the commercialized live attenuated vaccine FluMist® recommended for use between 2015 and 2016. FluMist® consisted of four attenuated influenza virus strains, including two attenuated Influenza B virus strains, an H3N2 (Switzerland/9715293/2013) and an H1N1 (California/7/2009 pandemic virus) attenuated strain. Mice were immunized with two attenuated vaccines in the same amount and the same amount of HK68/H3 virus was used as a challenge. The results showed that the immuno-protective effect of W7-791 was superior to the immunological effect of FluMist® (FIG. 88). As can be seen from the above study, one inoculation and immunization with W7-791, can provide very effective cross-immunological protection for the body.

### Example 6

### W7-791 can Simultaneously Trigger Effective Humoral Immunity and Cellular Immunity Responses

The influenza virus specific antibodies or virus neutralizing antibodies in the serum of immunized mice can be determined with an influenza virus hemagglutination inhibition test or virus neutralizing experiment. The results of the test of antibodies in mice showed that mice immunized with W7-791 only produced WSN virus-specific antibodies, but not against PR8 virus, HK68 (H3N1) and Wis (H3N2) viruses (as shown in FIG. 9, Panels A-C). When the serum in mice immunized with W7-791 was adopted to non-immunized mice and when such mice were infected with a variety of viruses, the serum from the immunized mice court only provide protection against WSN itself and could not provide mice with protection against infection from other viruses (FIG. 9, Panels D-F). This showed that humoral immunity was not the only source for the W7-791 virus strain to provide immunity.

The T lymphocytes of mice immunized with W7-791 were adaptively transferred to non-immunized mice and then such mice were infected with a variety of wild-type influenza viruses. The immunity provided by the adopted T lymphocytes for the mice was observed, thus determining the role played by T lymphocytes in vaccination protection. We found that after the T cells from mice immunized with W7-791 were adaptively transferred to non-immunized mice, it enabled such mice to partially receive broad-spectrum protection, thereby reducing the severity and symptoms of the disease when such mice were infected with various influenza viruses (FIG. 9, Panels D-F). This showed W7-791 could effectively induce a protective T cell immune response, in the body which is also consistent with the characteristics of live attenuated influenza virus vaccines.

### Example 7

### One Immunization with W7-791 can Effectively Protect Ferrets Against Infections by Different Influenza Viruses

Ferrets are currently considered to be a better model of influenza virus infection. To further investigate and confirm the effectiveness of W7-791 as a live attenuated influenza virus vaccine, we tested the immuno-protective effect W7-791 for ferrets. First, to assess W7-791 infection and virulence in ferrets, we respectively infected ferrets with 10⁶, 10⁷ and 10⁸ TCID₅₀ of W7-791 vaccine strains of virus and then observed the flu symptoms caused by the virus. We found that a dose of 10⁸ TCID₅₀ of W7-791 did not result in rising body temperatures and other flu symptoms in ferrets, which indicated that W7-791 was equally safe for ferrets as it was for mice (FIG. 10, Panels A and B). We then examined the immune antibody levels W7-791 in ferrets and found a significant increase in vaccine-specific antibodies in ferrets (FIG. 10, Panel C). Results of a hemagglutination inhibition test showed that these antibodies can bind with HA of WSN but cannot bind with HA of HK68/H3 or H5N1 virus (FIG. 10, Panel D). Four weeks after ferrets were immunized with W7-791, we perform the respective challenges with 10⁶ TCID₅₀ of WSN and 10⁶ TCID₅₀ of HK68/H3 viruses. The results showed that, compared to non-immunized animals, for ferrets immunized with 10³ and 10^{4.7} TCID₅₀ of W7-791, two days after such challenges, basically no virus could be detected in such ferrets (FIG. 10, Panels E-F). In addition, after such challenges, the flu related symptoms presented by such ferrets were also significantly lighter (FIG. 10, Panels G-H).

All scientific and technical terms used in this application have meanings commonly used in the art unless otherwise specified.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments of the present invention, the subject is a mammal. In some embodiments of the present invention, the subject is a human.

The use of the singular can include the plural unless specifically stated otherwise. As used in the specification and the appended claims, the singular forms “a”, “an”, and “the” can include plural referents unless the context clearly dictates otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

The phrase “comprises, consists essentially of, or consists of” is used as a tool to avoid excess page and translation fees and means that in some embodiments the given thing at issue comprises something, and in some embodiments the given thing at issue consists of something. For example, the sentence “In some embodiments, the composition comprises, consists essentially of, or consists of A” is to be interpreted as if written as the following two separate sentences: “In some embodiments, the composition comprises A. In some embodiments, the composition consists essentially of A. In some embodiments, the composition consists of A.” Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.”

Throughout the instant specification, drawings, and claims, a feature of an inventive embodiment may be discussed alone or in specific combination with another feature. The discussion of a given feature by itself or as a specific combination of features is not to be construed as limiting. Instead, embodiments of the present invention having the given feature alone and in combination with one or more other features are contemplated herein as if explicitly recited herein to the extent possible, e.g., except where the features are mutually exclusive, the given feature cannot be combined with the other feature, etc. For example, where Embodiment A discusses the presence of Feature 1, Embodiment B discusses Features 2 and 3, but no embodiment explicitly sets forth the combination of Features 1, 2, and 3, an embodiment comprising the combination of Features 1, 2, and 3 is contemplated herein as though the specific combination was explicitly recited so long as Features 1, 2, and 3 are combinable.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein but is only limited by the following claims.

1. A recombinantly engineered influenza virus which comprises an insertional mutation in a genomic segment that encodes an M2 protein.

2. The recombinantly engineered influenza virus of claim 1, wherein the genomic segment is segment 7 of a virus belonging to Influenza A virus or Influenza B virus.

3. The recombinantly engineered influenza virus of claim 1, wherein the genomic segment 6 of a virus belonging to Influenza C virus or Influenza D virus.

4. The recombinantly engineered influenza virus of claim 1, wherein the M2 protein is a consensus or wildtype sequence of a M2 protein of Influenza A virus, a consensus or wildtype sequence of a BM2 protein of Influenza B virus, a consensus or wildtype sequence of a CM2 protein of Influenza C virus, or a consensus or wildtype sequence of a DM2 protein of Influenza D virus.

5. The recombinantly engineered influenza virus of claim 1, wherein the insertional mutation encodes an amino acid segment that comprises or consists of 4-6 amino acid residues of SEQ ID NO: 6.

6. The recombinantly engineered influenza virus of claim 1, wherein the insertional mutation encodes an amino acid segment that comprises or consists of 4-6 contiguous amino acid residues of SEQ ID NO: 6.

7. The recombinantly engineered influenza virus of claim 1, wherein the insertional mutation encodes an amino acid segment that comprises or consists of SEQ ID NO: 6.

8. The recombinantly engineered influenza virus of claim 1 wherein the insertional mutation comprises or consists of SEQ ID NO: 7, wherein each “n” are independently any nucleotide.

9. The recombinantly engineered influenza virus of claim 1, wherein the insertional mutation comprises or consists of SEQ ID NO: 5.

10. The recombinantly engineered influenza virus of claim 1, wherein the insertional mutation is located at or within about 1-5 amino acid residues, of the C-terminal end of the ectodomain of the M2 protein.

11. The recombinantly engineered influenza virus of claim 1, wherein the insertional mutation is located at or within about 1-5 amino acid residues, of the N-terminal end of the transmembrane domain of the M2 protein.

12. The recombinantly engineered influenza virus of claim 1, wherein the insertional mutation is located at the cytoplasmic portion of the ion channel formed by the M2 protein.

13. The recombinantly engineered influenza virus of claim 1, wherein the M2 protein is of a virus belonging to Influenza A virus, and the virus is an H1, H2, H3, H5, H6, H7, H9, or H10 subtype and/or an H1, H2, or H3 subtype.

14. The recombinantly engineered influenza virus of claim 1, wherein the M2 protein is of a virus belonging to Influenza A virus, and the virus is an H1N1, H2N1, H3N1, H5N1, H6N1, H7N1, H9N1, H10N1, H1N2, H2N2, H3N2, H5N2, H6N2, H7N2, H9N2, H10N2, H1N6, H2N6, H3N6, H5N6, H6N6, H7N6, H9N6, H10N6, H1N7, H2N7, H3N7, H5N7, H6N7, H7N7, H9N7, H10N7, H1N8, H2N8, H3N8, H5N8, H6N8, H7N8, H9N8, H10N8, H1N9, H2N9, H3N9, H5N9, H6N9, H7N9, H9N9, or H10N9 subtype.

15. The recombinantly engineered influenza virus of claim 1, wherein the M2 protein comprises or consists of an amino acid sequence having at least 85% sequence identity to SEQ ID NO: 12, wherein each X are independently any amino acid residue.

16. The recombinantly engineered influenza virus of claim 1, wherein the M2 protein comprises an amino acid sequence having at least 85% sequence identity to SEQ ID NO: 2.

17. The recombinantly engineered influenza virus of claim 1, wherein the genomic segment encodes a mutated M2 protein that comprises of an amino acid sequence having at least 85% sequence identity to SEQ ID NO: 4.

18. A composition comprising a recombinantly engineered influenza virus according to claim 1.

19. The composition according to claim 18, and further comprising a pharmaceutically acceptable carrier and/or an adjuvant.

20. A kit comprising a recombinantly engineered influenza virus according to claim 1 packaged together with a drug delivery device.


SEQUENCE LISTING (12 sequences)

SEQ ID NO: 1 - DNA, 292 bp, Influenza A virus
  CDS (1)..(291)
atgagtcttctaaccgaggtcgaaacgcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgccttaaatacggtttgaaaagagggccttctacggaaggagtgccagagtctatgagggaagaatatcgaaaggaacagcagaatgctgtggatgttgacgatggtcattttgtcaacatagagctggagt

SEQ ID NO: 2 - protein, 97 aa, Influenza A virus
MSLLTEVETPIRNEWGCRCNDSSDPLVIAANIIGILHLILWILDRLFFKCIYRRLKYGLKRGPSTEGVPESMREEYRKEQQNAVDVDDGHFVNIELE

SEQ ID NO: 3 - DNA, 307 bp, Artificial Sequence
  Mutated Influenza A M2 sequence
  CDS (1)..(306)
atgagtcttctaaccgaggtcgaaacgcctatcagaaacgaatgggggtgcagatgcaacgattcaagtgatcctcgtcattgcggccgcatcgtcattgcagcaaatatcattggaatcttgcacttgatattgtggattcttgatcgtctttttttcaaatgcatttatcgtcgccttaaatacggtttgaaaagagggccttctacggaaggagtgccagagtctatgagggaagaatatcgaaaggaacagcagaatgctgtggatgttgacgatggtcattttgtcaacatagagctggagt

SEQ ID NO: 4 - protein, 102 aa, Artificial Sequence
  Synthetic Construct
MSLLTEVETPIRNEWGCRCNDSSDPRHCGRIVIAANIIGILHLILWILDRLFFKCIYRRLKYGLKRGPSTEGVPESMREEYRKEQQNAVDVDDGHFVNIELE

SEQ ID NO: 5 - DNA, 15 bp, Artificial
  Insertional mutation
gtcattgcggccgca

SEQ ID NO: 6 - protein, 6 aa, Artificial Sequence
  Insertional mutation
RHCGRI

SEQ ID NO: 7 - DNA, 15 bp, Artificial Sequence
  Random insert based on Influenza A virus
  misc_feature (1)..(5): n is a, c, g, or t
nnnnntgcggccgca

SEQ ID NO: 8 - DNA, 20 bp, Artificial Sequence
  Forward primer for M sequence
agcaaaagcaggtagatatt

SEQ ID NO: 9 - DNA, 20 bp, Artificial Sequence
  Forward primer for M sequence
ggggccaaagaaatagcact

SEQ ID NO: 10 - DNA, 20 bp, Artificial Sequence
  Forward primer for M sequence
tcctagctccagtgctggtc

SEQ ID NO: 11 - DNA, 10 bp, Artificial Sequence
  Vic-labeled insertion-specific mini-primer
tgcggccgca

SEQ ID NO: 12 - protein, 97 aa, Artificial Sequence
  M2 protein
  misc_feature (21)..(21): Xaa can be any naturally occurring amino acid
  misc_feature (26)..(27): Xaa can be any naturally occurring amino acid
  misc_feature (38)..(40): Xaa can be any naturally occurring amino acid
  misc_feature (55)..(55): Xaa can be any naturally occurring amino acid
  misc_feature (61)..(61): Xaa can be any naturally occurring amino acid
  misc_feature (70)..(70): Xaa can be any naturally occurring amino acid
  misc_feature (82)..(82): Xaa can be any naturally occurring amino acid
  misc_feature (86)..(86): Xaa can be any naturally occurring amino acid
  misc_feature (93)..(93): Xaa can be any naturally occurring amino acid
  misc_feature (96)..(97): Xaa can be any naturally occurring amino acid
MSLLTEVETPIRNEWGCRCNXSSDPXXIAANIIGILHXXXWILDRLFFKCIYRRXKYGLKXGPSTEGVPXSMREEYRKEQQXAVDXDDGHFVXIEXX